(19) United States
(12) Patent Application Publication
(10) Pub. No.: US 2025/0276188 A1
(43) Pub. Date: Sep. 4, 2025

(54) METHOD FOR CONTROLLING A MEDICAL DEVICE IMPLANTED IN A PATIENT IN PRESENCE OF AN EXTERNAL DEVICE IN THE PATIENT’S ENVIRONMENT

(71) Applicant: BIO TRONIK SE & Co. KG, Berlin (DE)

(72) Inventors: Thomas DOERR, Berlin (DE); Volker LANG, Berlin (DE); Bjoern DIEM, Berlin (DE)

(73) Assignee: BIO TRONIK SE & Co. KG, Berlin (DE)

(21) Appl. No.: 18/858,087

(22) PCT Filed: Apr. 18, 2023
(86) PCT No.: PCT/EP2023/059976

§ 371 Date: Oct. 18, 2024

(30) Foreign Application Priority Data
Apr. 28, 2022 (EP) 22170528.8

(51) Int. Cl. A61N 1/372

(52) U.S. Cl. CPC A61N 1/37223

(57) ABSTRACT

A method for controlling a medical device implanted in a patient in presence of an external device in the patient's environment includes: receiving a detection signal indicating the presence of the external device in the patient's environment, wherein the detection signal incudes a unique identifier for the external device; selecting an operating mode for the medical device by comparing the identifier to a list of known identifiers, wherein each known identifier is associated with a predefined operating mode for the medical device; and applying the selected operating mode to the medical device, thereby adjusting the medical device so that undesirable electromagnetic interaction between the medical device and the external device is reduced.

### CROSS-REFERENCE TO RELATED APPLICATIONS

This application is the United States National Phase under 35 U.S.C. § 371 of PCT International Patent Application No. PCT/EP2023/059976, filed on Apr. 18, 2023, which claims the benefit of European Patent Application No. 22170528.8, filed on Apr. 28, 2022, the disclosures of which are hereby incorporated by reference herein in their entireties.

### TECHNICAL FIELD

The present invention relates to a computer-implemented method for controlling a medical device, which is implanted in a patient, in presence of an external device in the patient's environment. Furthermore, the present invention relates to a control unit, a computer program and a computer-readable medium for carrying out the method and to an implantable medical device comprising said control unit.

### BACKGROUND

The presence of an external device in the environment of a patient carrying an implantable medical device may cause undesirable electromagnetic interference. This may have a negative impact on diagnostic and/or therapeutic functions of the implantable medical device and, in some cases, may even lead to the patient no longer being able to work in certain environments.

It is further known to control a machine based on information which is exchanged between the machine and an implantable medical device.

For example, U.S. Publication No. 2011/0092802 A1 describes a control device for controlling a machine, e.g., a vehicle, based on data received from an implant in dependence on the fitness of the implant wearer.

The present disclosure is directed toward overcoming one or more of the above-mentioned problems, though not necessarily limited to embodiments that do.

### SUMMARY

It may be seen as an objective of the present invention to provide an improved method for controlling a medical device, which is implanted in a patient, in presence of an external device in the patient's environment. Another objective of the present invention may be to provide a control unit, a computer program and a computer-readable medium for carrying out said method as well as an implantable medical device comprising said control unit.

At least these objectives may be achieved by the subject-matter of the independent claims. Advantageous embodiments are defined in the dependent claims as well as in the corresponding specification and figures.

A first aspect of the present invention relates to a computer-implemented method for controlling a medical device, which is implanted in a patient, in presence of an external device in the patient's environment. The method comprises at least the following steps: receiving a detection signal indicating the presence of the external device in the patient's environment, wherein the detection signal comprises a unique identifier for the external device; selecting an operating mode for the medical device by comparing the identifier to a list of known identifiers, wherein each known identifier is associated with a predefined operating mode for the medical device; and applying the selected operating mode to the medical device, thereby adjusting the medical device so that undesirable electromagnetic interaction between the medical device and the external device is reduced.

The method may be carried out automatically by a processor.

Electromagnetic interaction, also referred to as electromagnetic interference (EMI), may be understood as a disturbance generated by the external device that may affect electrical and/or electronic components of the medical device by electromagnetic induction, electrostatic coupling and/or conduction (and vice versa). The disturbance, which may be caused by changing electrical currents and voltages, may degrade the performance of these components or even stop them from functioning.

The detection signal may be seen as an information about a spatial proximity of the external device with respect to the medical device. The detection signal may have been generated by and sent from the external device, e.g., one or more detection units of the external device, to the medical device. Such a detection unit may be a transmitter, e.g., a radio beacon, or a transceiver, e.g., an active or passive transponder. In other words, the detection signal may be received via a unidirectional or bidirectional communication link. The detection signal may be received via a wireless communication link and/or the Internet. The detection signal may be received directly from the external device or indirectly via at least one additional data communication device (e.g., a smartphone, smartwatch, tablet, laptop, PC or server) which interconnects the medical device and the external device for data communication.

The identifier may be seen as a unique predetermined code associated with one external device or with one group of identical or similar external devices. Thus, each known identifier in the list may stand for one (known) external device or one group of (known) identical or similar external devices.

The list of known identifiers may be stored in a memory of the medical device. For example, the list may comprise more than 10, in particular more than 100 or even more than 1,000 or more than 10,000 known identifiers.

Each predefined operating mode may be defined by a set of specific operating parameters, wherein different predefined operating modes in the list may differ from each other in at least one of these parameters.

Different known identifiers in the list may be associated with different or identical predefined operating modes.

The selected operating mode may be selected by searching in the list of known identifiers for an item which is identical to the identifier encoded by the detection signal and by selecting the predefined operating mode associated with this item.

The method helps to ensure safe operation of active implants in the presence of external devices, such as machines, industrial plants or vehicles, especially in professional environments, in an efficient and reliable manner without the need of manual interaction. Thus, possible limitations for patients carrying active implants and working in potentially hostile environments, e.g., in the vicinity of a powerful electric machine, can be at least partially eliminated.

A second aspect of the present invention relates to a control unit comprising a processor configured to carry out the method as described above and below. The control unit may include hardware and/or software modules. In addition to the processor, the control unit may include a memory and data communication interfaces for data communication with peripheral devices, e.g., an external device as described above and below.

The control unit may be part of an implantable medical device. However, it is also possible that the control unit is integrated in a user device (e.g., a remote, smartphone, smartwatch, tablet, laptop or PC) connected to the implantable medical device for data communication. For example, the (external) user device may be configured to run an application for controlling one or more functions of the implantable medical device remotely.

A third aspect of the present invention relates to an implantable medical device comprising the control unit as described above and below. The implantable medical device may be, for example, an implantable pulse generator (IPG), an implantable cardioverter defibrillator (ICD), a cardiac resynchronization therapy (CRT) pacemaker, a neurostimulator, an electrocardiogram (ECG) recorder, a pressure sensor, a biochemical sensor, a drug pump or a hearing aid.

Further aspects of the present invention relate to a computer program comprising instructions which, when the program is executed by a processor, cause the processor to carry out the method as described above and below and to a computer-readable medium in which the computer program is stored. The computer program may be executed by a processor of the control unit.

The computer-readable medium may be a volatile or non-volatile data storage device. For example, the computer-readable medium may be a hard drive, USB (universal serial bus) storage device, RAM (random-access memory), ROM (read-only memory), EPROM (erasable programmable read-only memory) or flash memory. The computer-readable medium may also be a data communication network for downloading program code, such as the Internet or a data cloud.

It should be noted that features of the method as described above and below may be features of the control unit, the computer program and the computer-readable medium, and vice versa.

Embodiments of the present invention may be considered, without limiting the present invention, as being based on the ideas and findings described below.

According to an embodiment, the list may comprise known identifiers for different device types. Additionally or alternatively, the list may comprise known identifiers for at least one of the following device types: an industrial machine, an industrial tool, an industrial robot, a vehicle (e.g., a car, truck, bus or motorcycle), in particular an electric vehicle, a security gate, a further medical device (e.g., an MRI system). This allows the medical device to be automatically adapted to (completely) different device types without the need of reprograming the medical device.

According to an embodiment, by applying the selected operating mode, at least one diagnostic and/or therapeutic function of the medical device may be modified or deactivated. Such a function may be configured, for example, to provide electric pulses in a controlled manner, e.g., in order to stimulate the patient's heart, spine or brain, and/or to monitor (electrical) biosignals from the patient. Thus, modifying the function(s) may comprise, for example, modifying the form and/or duration of these electric pulses and/or modifying one or more filters for filtering the biosignals. This embodiment may significantly reduce the risk of EMI-related inaccuracies, or even malfunctions, when operating the medical device near the external device.

The medical device may be configured to sense biosignals from the patient along different sensing vectors. According to an embodiment, applying the selected operating mode may cause the medical device to switch between these sensing vectors, for example, from one or more currently active sensing vectors to one or more alternative sensing vectors which may be less susceptible to EMI in the presence of the external device. For example, each sensing vector may be defined by a pair of electrodes in contact with the patient's body in dependence on their position relative to the patient's body and their mutual distance. Such an electrode may be, for example, part of an implantable lead or implantable housing of the medical device. This makes it possible to reduce EMI without having to interrupt or completely deactivate sensing of the biosignals.

According to an embodiment, by applying the selected operating mode, at least one hardware component of the medical device, in particular at least one sensor of the medical device, may be deactivated. This may be done, for example, by disconnecting the hardware component(s) from a battery of the medical device or by ignoring an output signal provided by the hardware component(s). The sensor(s) may be configured to sense (electrical) biosignals from the patient and/or magnetic fields.

The medical device may comprise an interference detector configured to detect interference in an output signal provided by at least one sensor of the medical device. The sensor(s) may be configured to sense (electrical) biosignals from the patient and/or magnetic fields. According to an embodiment, by applying the selected operating mode, the interference detector may be modified or deactivated. This may significantly reduce inaccuracies and/or malfunctions of the interference detector caused by EMI, in particular in the presence of very powerful electrical machines such as electric vehicles or industrial robots.

According to an embodiment, applying the selected operating mode may prevent the medical device from switching into a power saving mode. This ensures that the medical device is fully functional in the presence of the external device.

The medical device may comprise a recording function configured to record an output signal provided by at least one sensor of the medical device. The sensor(s) may be configured to sense (electrical) biosignals from the patient and/or magnetic fields. According to an embodiment, applying the selected operating mode may cause the recording function to:
start recording the output signal and/or mark new recordings of the output signal as being recorded in presence of the external device; or
stop recording the output signal.

For example, the (new) recordings may be used to identify undesirable electromagnetic interference in the presence of the external device. Such information may be used to further improve the predefined operating modes and/or to define new operating modes for the medical device. Deactivating the recording function may help to further reduce undesirable electromagnetic interference.

According to an embodiment, the selected operating mode may be applied only as long as the detection signal is received. In other words, the medical device may automatically be switched from the selected operating mode back into a previous operating mode when the detection signal is no longer received, e.g., when a period of time since the detection signal has been last received is longer than 1 s, 10 s or 1 min. This enables automatic activation and deactivation of the corresponding predefined operating mode without additional interaction, e.g., by the patient or a physician.

According to an embodiment, the detection signal may be received via a wireless data communication link for data communication between the medical device and the external device, in particular unidirectional data communication. For example, the detection signal may be received via Bluetooth (e.g., Bluetooth Low Energy), Wi-Fi, LoRaWAN (low-power wide-area network), WPAN, ZigBee, ISM (industrial, scientific and medical radio), MICS (Medical Implant Communication Service), a cellular network (e.g., GSM, LTE or 5G), near-field communication (NFC), ultra-wideband or a combination of at least two of these examples.

It may be that the detection signal is transmitted in regular time intervals by a transmitter or transceiver of the external device within a specific range of, for example, 100 m or less, 10 m or less or 1 m or less. A receiver or transceiver integrated in the medical device may then receive the detection signal as soon as the patient carrying the medical device is within said range. This has the advantage that no first (interrogating) signal has to be sent from the medical device to the external device prior to receiving the detection signal. This may improve energy efficiency of the medical device and reduce manufacturing costs.

Alternatively, the method may comprise a step of sending a first signal from the medical device to the external device, wherein the first signal may cause the external device to send the detection signal as a second signal.

According to an embodiment, the detection signal may be received via Bluetooth, for example, using Bluetooth Low Energy (BLE) technology. This enables efficient and reliable short-range data communication between the medical device and the external device.

It should be noted that possible features and advantages of embodiments of the present invention are described above and below partly with reference to a method for controlling an implantable medical device and partly with reference to a control unit specifically designed for carrying out said method. A person skilled in the art will recognize that the features described for individual embodiments may be suitably transferred to other embodiments in an analogous manner, may be adapted and/or interchanged to arrive at further embodiments of the present invention and possibly synergistic effects.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Advantageous embodiments of the present invention are further explained below with reference to the accompanying drawings. Neither the drawings nor the description are to be construed as limiting the present invention.

FIG. 1 shows a patient carrying an implantable medical device according to an embodiment of the present invention in the vicinity of an industrial robot.

FIG. 2 shows the implantable medical device of FIG. 1 in more detail.

### DETAILED DESCRIPTION

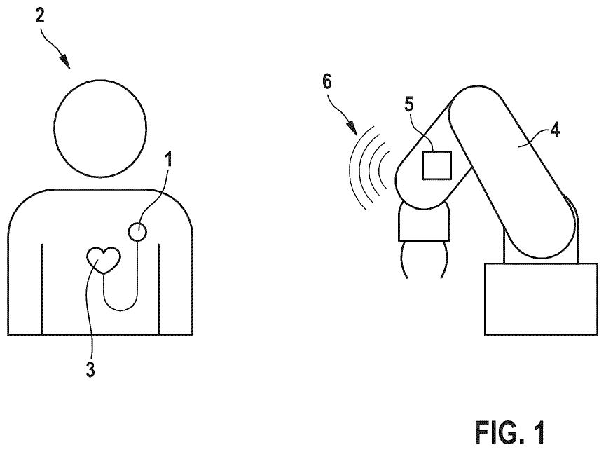

FIG. 1 shows a medical device 1 implanted in the body of a patient 2. For example, the medical device 1 may be adapted to generate electrical signals for stimulating the patient's heart 3 and/or to monitor electrical biosignals from the patient's heart 3. The patient 2 is near an external device 4, here an industrial robot.

The medical device 1 and the external device 4 may be interconnected for data communication via a wireless communication link (unidirectional or bidirectional), for example, via Bluetooth. However, all low-power communication methods are, in principle, suitable for data communication between the two devices 1, 4.

The external device 4 may comprise one or more detection units 5, e.g., one or more radio beacons (for unidirectional data communication) and/or one or more active and/or passive transponders (for bidirectional data communication), for transmitting via the wireless communication link a detection signal 6 which encodes a unique identifier for the external device 4. For example, the detection unit 5 may be a Bluetooth low energy (BLE) beacon in the form of an asset tag attached to the external device 4, here a moving arm of the industrial robot.

In addition, the medical device 1 may be configured for encrypted data communication with the external device 4.

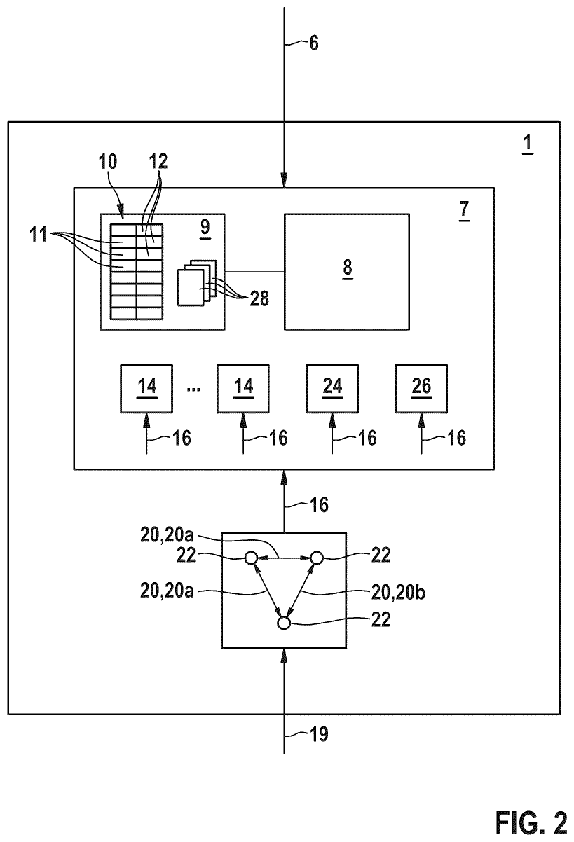

As shown in FIG. 2, the medical device 1 may comprise a control unit 7 with a processor 8 and a memory 9. The processor 8 may be configured to carry out a method for controlling the medical device 1 by executing instructions of a computer program which may be stored in the memory 9. The method is described in more detail below.

In a first step, the control unit 7 receives the detection signal 6. This may be the case when the patient 2, and thus the control unit 7, is within the range of the detection unit 5, for example, when the distance of the control unit 7 from the detection unit 5 is 10 m or less or 5 m or less.

As shown here, the control unit 7 may receive the detection signal 6 directly from the detection unit 5.

However, it is also possible for the detection signal 6 to be transmitted via at least one additional relay device which connects the medical device 1 to the external device 4 for data communication. The additional relay device may be, for example, a cloud or network instance which allows data communication only with valid authentication of the medical device 1 and/or the external device 4. This further increases data security.

In a second step, the control unit 7 selects an operating mode for the medical device 1 based on the detection signal 6. This may be done by comparing the identifier encoded by the detection signal 6 with a list 10 of known identifiers 11, wherein each known identifier 11 is associated with a predefined operating mode 12 for the medical device 1. The list 10 may be stored in the memory 9. The selected operating mode 12 may be a predefined operating mode 12 whose known identifier 11 is identical to the identifier encoded by the detection signal 6.

In a third step, the control unit 7 applies the selected operating mode 12 to the medical device 1, thereby changing one or more operating parameters of the medical device 1 in such a manner that undesirable electromagnetic interference between the medical device 1 and the external device 4 is avoided or at least reduced. The selected operating mode 12 may have been individually adapted to the external device 4 by programming the medical device 1 accordingly.

The method may additionally comprise a fourth step of switching the medical device 1 from the selected operating mode 12 back to a previous operating mode when the detection signal 6 is no longer received, e.g., when a period of time since the detection signal 6 has been last received is longer than 1 s, 10 s or 1 min.

For example, the list 10 may comprise known identifiers 11 for different industrial robots.

It is also possible that the list 10 comprises known identifiers 11 for different types of external devices 4, such as industrial robots, industrial machines, industrial tools, (electric) vehicles, security gates or further medical devices (e.g., MRI systems).

For example, the control unit 7 may comprise one or more diagnostic and/or therapeutic functions 14 which may be configured to process an output signal 16 of one or more sensors 18 in contact with the patient's body and to control one or more active elements of the medical device 1 accordingly (the active elements may include the sensor(s) 18). The function(s) 14, as well as other functions of the medical device 1 (see below), may be software and/or hardware modules of the control unit 7.

By applying the selected operating mode 12, one or more diagnostic and/or therapeutic functions 14 may be modified or deactivated and, additionally, one or more other diagnostic and/or therapeutic functions 14 may be activated.

The output signal 16 may encode an electrical biosignal 19 received from the patient 2 as input signal, wherein the biosignal 19 may have been sensed by the sensor(s) 18 along different sensing vectors 20. The position, orientation and length of each sensing vector 20 may be defined by a pair of electrodes 22 which are in contact with different portions of the patient's body.

Additionally or alternatively, the sensor(s) 18 may be adapted to sense magnetic fields in the patient's environment.

For example, by applying the selected operating mode 12, at least one currently active sensing vector 20a may be blocked and instead at least one alternative sensing vector 20b less susceptible to EMI may be activated and used to sense the biosignal 19.

Alternatively, the sensor(s) 18 may be completely deactivated by applying the selected operating mode 12.

The control unit 7 may additionally comprise an interference detector 24 configured to detect interference in the output signal 16. Applying the selected operating mode 12 may modify the interference detector 24 so that certain interference patterns in the output signal 16 are temporarily ignored by the interference detector 24. Alternatively, applying the selected operating mode 12 may completely deactivate the interference detector 24.

The interference detector 24 may, for example, be configured to detect the presence of an MRI system as the external device 4 based on interference in the output signal 16, wherein the control unit 7 may be configured to switch the medical device 1 into a special operating mode when the MRI system is detected.

In addition, the control unit 7 may comprise a recording function 26 configured to record the output signal 16 and/or to store recordings 28 of the output signal 16 in the memory 9. Applying the selected operating mode 12 may cause the recording function 26 to start recording the output signal 16 and/or to mark new recordings 28 as being recorded in presence of the external device 4. Alternatively, recording of the output signal 16 may be stopped by applying the selected operating mode 12.

For example, in the case of a loop recorder (biomonitor), episode recording may be enabled or disabled, or episode recordings based on, for example, P wave detection may be temporarily suspended to avoid inadequate recordings and/or EMI.

Additionally or alternatively, applying the selected operating mode 12 may prevent the medical device 1 from switching into a power saving mode. For example, night reduction may be deactivated during shift work or in an active phase of the patient 2 during on-call duty.

In other words, the medical device 1 may automatically modify its operating mode upon detection of a registered external device 4. As pointed out above, such automatic modification may include, for example:
modification of an interference signal detection or an interference mode of the medical device 1, for example, when a powerful electric vehicle is detected as the external device 4;
activation and/or deactivation of at least one diagnostic and/or therapeutic function 14 of the medical device 1;
activation and/or deactivation of at least one technical component, in particular at least one sensor 18 (e.g., a magnetic field sensor), of the medical device 1;
activation and/or deactivation of at least one function and/or algorithm which could cause an undesirable interaction with the external device 4, for example, impair proper operation of a machine by the patient 2 (for example, in the case of an implantable pulse generator, night reduction may be deactivated).

For example, in order to detect a vehicle as the external device 4, the medical device 1 may exchange BLE pairing information with an entertainment system of the vehicle, e.g., an audio system.

It should be noted that, in the claims, the word “comprising” does not exclude other elements or steps, and the indefinite article “a” or “an” does not exclude a plurality. A single processor or controller or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope of the claims.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this present invention.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A computer-implemented method for controlling a medical device implanted in a patient in presence of an external device in the patient's environment, the method comprising:
receiving a detection signal indicating the presence of the external device in the patient's environment, wherein the detection signal comprises a unique identifier for the external device;
selecting an operating mode for the medical device by comparing the identifier to a list of known identifiers, wherein each known identifier is associated with a predefined operating mode for the medical device; and
applying the selected operating mode to the medical device, thereby adjusting the medical device so that undesirable electromagnetic interaction between the medical device and the external device is reduced,
wherein the list comprises known identifiers for different device types; and/or
wherein the list comprises known identifiers for at least one of the following device types: an industrial machine, an industrial tool, an industrial robot, a vehicle, a security gate, a further medical device.

2. The method of claim 1, wherein each predefined operating mode may be defined by a set of specific operating parameters, wherein different predefined operating modes in the list may differ from each other in at least one of these parameters.

3. The method of claim 1,
wherein, by applying the selected operating mode, at least one diagnostic and/or therapeutic function of the medical device is modified or deactivated.

4. The method of claim 1,
wherein the medical device is configured to sense biosignals from the patient along different sensing vectors;
wherein applying the selected operating mode causes the medical device to switch between the sensing vectors.

5. The method of claim 1,
wherein, by applying the selected operating mode, at least one hardware component of the medical device, in particular at least one sensor of the medical device, is deactivated.

6. The method of claim 1,
wherein the medical device comprises an interference detector configured to detect interference in an output signal provided by at least one sensor of the medical device;
wherein, by applying the selected operating mode, the interference detector is modified or deactivated.

7. The method of claim 1,
wherein applying the selected operating mode prevents the medical device from switching into a power saving mode.

8. The method of claim 1,
wherein the medical device comprises a recording function configured to record an output signal provided by at least one sensor of the medical device;
wherein applying the selected operating mode causes the recording function to:
start recording the output signal and/or mark new recordings of the output signal as being recorded in presence of the external device; or
stop recording the output signal.

9. The method of claim 1,
wherein the selected operating mode is applied only as long as the detection signal is received.

10. The method of claim 1,
wherein the detection signal is received via a wireless data communication link for data communication, in particular unidirectional data communication, between the medical device and the external device.

11. The method of claim 1,
wherein the detection signal is received via Bluetooth.

12. A control unit comprising a processor configured to carry out the method of claim 1.

13. An implantable medical device comprising the control unit of claim 12.

14. The implantable medical device of claim 13, wherein the implantable medical device is at least one of an implantable pulse generator, an implantable cardioverter defibrillator, a cardiac resynchronization therapy GR pacemaker, a neurostimulator, an electrocardiogram recorder, a pressure sensor, a biochemical sensor, a drug pump or a hearing aid.

15. A computer program comprising instructions which, when the program is executed by a processor, cause the processor to carry out the method of claim 1.
